(19) United States
(12) Patent Application Publication
(10) Pub. No.: US 2009/0238332 A1
(43) Pub. Date: Sep. 24, 2009

(54) Breast x-ray machine

(75) Inventor: Marlene Rhonda Farrell-Trent, Brooklyn, NY (US)

Correspondence Address: MARLENE R. FARRELL-TRENT, P.O. BOX 23395, BROOKLYN, NY, 11202, US

(73) Assignee: MARLENE FARRELL-TRENT

(21) Appl. No.: 12/004,062

(22) Filed: Mar. 18, 2008

(51) Int. Cl. A61B 6/04

(52) U.S. Cl. 378/37

(57) ABSTRACT

This Breast X-ray Mammogram Machine was design for both women and men. Today we have just as much men with breast cancer as women. Therefore, with this new breast x-ray mammogram machine. You will be able to detect any abnormalities in both the breast and the chest area. This breast x-ray mammogram machine is a large rectangular x-ray mammogram machine. This machine would measure up to seven feet in height. However, this breast x-ray mammogram machine would have a horizontal metal base; a set of plastic/metal wheels, film holder for the x-ray film and x-ray components for a copper anode. A tungsten target and a cathode inside a glass housing with a lead casting. Included in the breast x-ray mammogram machine would be an AC motor.

### BACKGROUND OF THE INVENTION

The Breast X-ray Mammogram Machine, is a new and improve breast mammogram machine; from the one which is now in hospitals, clinics. The advantages The Breast X-ray Mammogram Machine have is cups these different cups will be the cup size of a women bra cup size. And was design for both women and men. The breast x-ray mammogram machine that is available now is for women only and don't have cups. And the technician have to press down on a bar so that bar can press down on a woman breast to take the picture for the x-ray; and it is very, very painful and the procedure last about (20) twenty minutes. And if the picture the technician taken is not readable; you have to do it all over again; and that means more pain for you. You also have to hug the mammogram machine so the technician can get a good picture.

### BRIEF SUMMARY OF THE INVENTION

The patient will step up onto the Non-slip platform; then the patient will lean face down onto the x-ray bed. Then the technician will help the patient place their breast into each cup. The patient will place their head onto the head rest; then both hands above their head; while bending over the machine. After which the technician will check to see if the patient is comfortable; once the patient tell the technician she or he is ready, the technician will take the x-ray. After the technician finish checking the x-ray and technician can see the x-ray is fine the patient can get dress and leave. By using the Breast X-Ray Mammogram Machine, we will see many patient going out and having their mammogram done. Recently I had my breast x-ray mammogram done with the mammogram that the hospital have now; and the amount of pain I had in my breast during and after was horrible and this pain lasted for about a week. Therefore, with my new and improve breast mammogram machine there will be no more pain when having our mammogram taken.

### BRIEF DESCRIPTION OF THE VIEWS OF THE DRAWING

FIG. 1—is a perspective overview of the drawing for the Breast X-ray Mammogram Machine.

FIG. 2—is a perspective overview of the various cups in FIG. 1 drawing of The Breast X-ray Mammogram Machine.

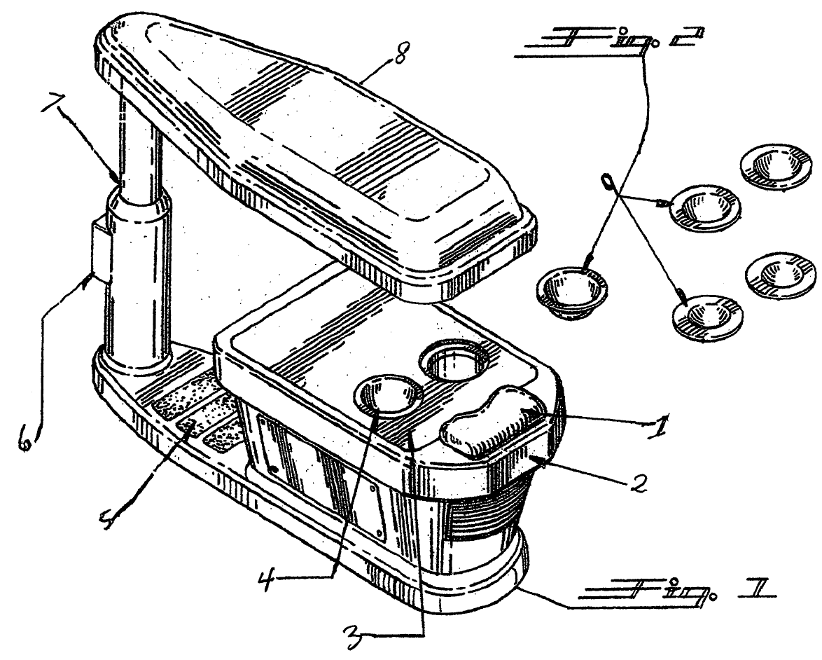

### DETAILED DESCRIPTION OF THE INVENTION

To facilitate one's comprehension and understanding of a new product, that idea must be describe not only as it exists as an answer to a subsistent problem. But also as it functions physically. Incorporated into this overview is a description of the invention's physical appearance.

Based upon the inventions' description and our outside research, we will describe the basic overall features, shapes and design of the Breast X-ray Mammogram Machine. Materials of which the invention would be produce will also be produce will also be described.

The design of the Breast X-rat Mammogram Machine; calls for materials such as plastic, metal, glass, electrical components and electronic components to be utilized as its major components.

The Breast X-ray Mammogram Machine would consist of a large, rectangular X-ray machine. The machine would measure up to seven feet in height.

The X-ray machine would have a horizontal metal base, a set of plastic/metal wheels, a film holder for X-ray film and X-ray components such as a copper anode, a tungsten target and a cathode inside a glass housing and a lead casing. Other features of The Breast X-ray Mammogram Machine would include an AC motor, an adjustable vertical support, a plastic switch or ac plastic button to move the Breast X-ray Mammogram Machine; upwards and downwards, a plastic switch to start the X-ray, an overhead canopy, two plastic cups for the patients breast and an insulated power cord with a plastic/metal plug. The plastic cups would be offered in various sizes or would be adjustable to suit the needs of different patients.

The plastic components of the Breast X-ray Mammogram Machine could be formed with the use of plastic molding techniques such as injection molding or blow molding. Injection molding is a process that has been in use since 1920s and provides a versatility almost unmatched in the mass production of any material. It is required that melted plastic be forcefully injected into relatively cool molds. As the plastic begins to harden, it takes on the shape of the mold cavity and, when cool, requires few post molding operations. Other advantages of this process include its speeds of production and its ability to allow multiple parts to be simultaneously molded.

Blow molding in the production of plastic shapes is a form of extrusion, a major technique in the plastic industry. Extrusion is used to push a molten tube, called a parison, into a bottle shape mold. Compressed air then forces the parison against the cold walls of the mold, hence the term “blow molding.”

Mold are generally side fed, with the thickness controlled by a tapered mandrel (core) or a variable-orifice die. Continuous extrusion of the plastic is possible through the use of multiple blow molds.

The Breast X-ray Mammogram Machine would be light blue in color.

To use the Breast X-RAY Mammogram Machine, a patient would bend over and have a technician fit her breast into the cups. The technician would then take an X-ray which could later be used for detecting an abnormalities in the breast or the chest area.

1. Something comprising elite:
The Breast X-ray mammogram machine to be use for taking X-ray of the breast comprising:
Head Rest is use for patients to lay head on:
X-ray bed this is where the X-ray is inserted:
Patient Lean Face Down onto X-Ray Bed and they will place their hands above there heads and have their X-ray taking:
Patient breast cup openings, the patient breast is place in each one of the cups:
Non Slip Platform, this platform is for the patients to stand on:
Control Panel for the mammogram machine:
Height Adjustment is for the technician to adjust the high of the canopy:
The Overhead Canopy:
Five various cups:
One drawing sheet.
The Breast X-ray Mammogram Machine is a large rectangular X-ray machine; this machine will measure up to seven feet in height and would have a horizontal metal base.
